(19) United States
(12) Patent Application Publication
(10) Pub. No.: US 2026/0245847 A1
(43) Pub. Date: Aug. 20, 2026

(54) METHODS FOR CALIBRATING A MASS SPECTROMETRY SYSTEM

(71) Applicant: INTABIO, LLC, FREMONT, CA (US)

(72) Inventors: Mariam ELNAGGAR, FREMONT, CA (US); Hongfeng YIN, FREMONT, CA (US)

(21) Appl. No.: 19/157,618

(22) PCT Filed: Feb. 14, 2024
(86) PCT No.: PCT/US2024/015814

§ 371 Date: Aug. 18, 2025

Related U.S. Application Data
(60) Provisional application No. 63/485,955, filed on Feb. 20, 2023.
(60) Provisional application No. 63/585,637, filed on Sep. 27, 2023.

(51) Int. Cl. H01J 49/00

(52) U.S. Cl. CPC H01J 49/0009

(57) ABSTRACT

The presently claimed and described technology generally relates to methods for calibrating an isoelectric focusing-mass spectrometry system. The method may include introducing an analyte mixture into a fluid channel, wherein the analyte mixture comprises at least one ampholyte and a cathodic spacer, wherein the cathodic spacer has a known mass spectral profile; applying an electric field across the fluid channel to separate the analyte mixture via isoelectric focusing; mobilizing the focused analyte misture; expelling the mobilized analyte mixture via electrospray ionization into a mass spectrometer from a single orifice; collecting a cathodic spacer mass spectrometric (MS) data comprising at least one mass spectrum from the cathodic spacer; comparing the cathodic spacer MS data with the known mass spectral profile of the cathodic spacer to generate a set of corresponding values; and adjusting one or more MS calibration parameters based on the set of corresponding values.

### RELATED APPLICATIONS

The present patent application claims the priority benefit of U.S. Provisional Patent Application Ser. No. 63/485,955, filed Feb. 20, 2023 and U.S. Provisional Patent Application Ser. No. 63/585,637, filed Sep. 27, 2023, the content of each is hereby incorporated by reference in its entirety into this disclosure.

### BACKGROUND

In isoelectric focusing, a sample (e.g., a biological sample, such as a protein sample), may be mixed with an ampholyte solution. This sample/ampholyte solution is then pushed into a channel (e.g., a capillary or a channel in a microfluidic device). An electric field is then applied across the channel to cause ion migration and, as a result, establish a pH gradient across the channel. At the same time, e.g., a protein sample will also migrate toward the region along the channel where the local pH is the closest to the protein's isoelectric point (pI). When the protein reaches its pI, it will have a net zero charge and will stop migrating. This step is called the “focusing” step. An image of the channel after all proteins in the sample reach their pI will show the relative abundance of the various protein species in the sample. The pI of the various protein species can be determined by their relative position to pI markers that are added to the sample solution.

While the imaged UV trace gives pI and relative quantitation of the various proteins in the sample, there is a need to be able to identify the protein using a mass spectrometer. This is achieved by mobilizing the proteins. During mobilization, the pH at the catholyte end starts to drop, and the protein will start to carry charges which causes it to migrate toward the mobilizer junction. Once it reaches the mobilizer junction, it will be carried by the flowing mobilizer out of the microfluidic chip and sprayed into a mass spectrometer interface. Coupling of capillary isoelectric focusing (cIEF) with downstream mass spectrometry can be a powerful tool for characterization of, e.g., biomolecules (such as proteins). Direct coupling of imaged cIEF (icIEF) with downstream mass spectrometry may provide a significant advantage by allowing the collection and correlation of icIEF data (e.g., UV data, information on isoelectric points (pI), quantitative information, etc.) and mass spectrometric (MS) data of an analyte. However, coupling a mass spectrometer to an additional upstream analysis mode might restrict direct access to the mass spectrometer and, thus, complicate the performance of such tasks as MS calibration.

Mass spectrometric calibration is necessary to obtain accurate MS data. When the mass spectrometer is coupled to another instrument, such as an isoelectric focusing instrument, MS calibration may require decoupling of the mass spectrometer to perform calibration (e.g., via infusion of MS calibrant), which is tedious and not viable during longer sequences of automated runs. The calibration may also be performed by introducing a mass calibrant through the upstream instrument (e.g., icIEF with an integrated ESI sprayer). However, such method also has its drawbacks, as, e.g., it results in longer sequences (by adding a separate run for the calibrant) and causes additional wear on the upstream system. While data files can be calibrated after the fact, this puts additional responsibility on a user to be diligent about additional processing, while not correcting the possible systemic problem of mass drift. Thus, there is a need for a more efficient way to calibrate integrated (e.g., icIEF-MS) systems.

### SUMMARY

In some aspects, the need for MS calibration on an isoelectric focusing-mass spectrometry system can be addressed by using a cathodic spacer used in the isoelectric focusing portion of the analysis.

One general aspect includes a method for calibrating an isoelectric focusing-mass spectrometry system. The method also includes introducing an analyte mixture into a fluid channel, where the analyte mixture includes at least one ampholyte and a cathodic spacer, where the cathodic spacer has a known mass spectral profile. The method also includes applying an electric field across the fluid channel to separate the analyte mixture via isoelectric focusing. The method also includes mobilizing the focused analyte mixture. The method also includes expelling the mobilized analyte mixture via electrospray ionization into a mass spectrometer from a single orifice. The method also includes collecting cathodic spacer mass spectrometric (MS) data, including at least one mass spectrum from the cathodic spacer. The method also includes comparing the cathodic spacer MS data with the known mass spectral profile of the cathodic spacer to generate a set of corresponding values. The method also includes adjusting one or more MS calibration parameters based on the set of corresponding values.

In some aspects, adjusting the one or more MS calibration parameters includes adjusting one or more calibration parameters for a mass spectrometer. In some aspects, adjusting the one or more MS calibration parameters includes adjusting one or more parameters for post-acquisition data recalibration.

In some aspects, the analyte mixture further includes a sample. In another aspect, the sample includes one or more biomolecules. In another aspect, adjusting the one or more MS calibration parameters includes adjusting one or more parameters for post-acquisition data recalibration.

In some aspects, the method further includes collecting a sample MS data includes at least one mass spectrum of the sample. In some aspects, the method further includes adjusting the sample MS data based on the adjusted one or more mass spectrometer calibration parameters.

In an aspect, the cathodic spacer MS data is collected as a separate data set from the sample MS data. In another aspect, the cathodic spacer MS data and the sample MS data are collected as part of a combined data set.

In an aspect, a portion of the combined data set is used for comparing the cathodic spacer MS data with the known mass spectral profile of the cathodic spacer. In another aspect, the cathodic spacer MS data and/or the combined data set is collected using a single mass range. In an aspect, the single mass range starts at a value above an m/z of at least one of the ampholytes. In an aspect, the cathodic spacer MS data and/or the combined data set is collected using two or more mass ranges. In another aspect, the two or more mass ranges include a first mass range and a second mass range, wherein the second mass range starts at a value above an m/z of at least one of the ampholytes; and wherein the first mass range starts at a lower value than the value at which the second mass range starts.

In an aspect, the single mass range or at least one of the two or more mass ranges starts at about 2000 m/z units. In another aspect, at least one of the two or more mass ranges starts at about 400 m/z units. In an aspect, at least one of the two or more mass ranges starts at about 0 m/z units.

In an aspect, the cathodic MS data is collected using different declustering potential, collision energy, and/or time bins to sum as compared to the sample MS data. In an aspect, the single orifice is in line with the fluid channel.

In an aspect, the method further includes correlating the separated analyte mixture peaks detected by imaging of the fluid channel or a portion thereof with an MS data for the separated analyte mixture.

In an aspect, the fluid channel or a portion thereof is imaged during or after the isoelectric focusing separation and/or mobilization.

In an aspect, the cathodic spacer forms multimers when subjected to electrospray ionization (ESI).

In an aspect, the cathodic spacer is a basic amino acid. In another aspect, the amino acid is arginine, histidine, or lysine. In a further aspect, the amino acid is arginine.

In an aspect, the cathodic spacer MS data includes at least one mass spectrum from one or more multimers of arginine.

In an aspect, the cathodic spacer MS data includes m/z peaks that have an intensity greater than about 800 counts per sec (cps), alternatively greater than about 900 cps, alternatively greater than about 1000 cps, alternatively greater than about 1100 cps, or alternatively greater than about 1200 cps.

In an aspect, the known mass spectral profile of the cathodic spacer includes two or more m/z peaks.

In an aspect, the method further includes excluding a direct injection of a calibrant into the mass spectrometer.

In an aspect, the method further includes adjusting the one or more mass spectrometer calibration parameters at least every other introduction.

A system of one or more computers can be configured to perform particular operations or actions by virtue of having software, firmware, hardware, or a combination of them installed on the system that, in operation, causes or cause the system to perform the actions. One or more computer programs can be configured to perform particular operations or actions by virtue of including instructions that, when executed by data processing apparatus, cause the apparatus to perform the actions.

One general aspect includes a computer-implemented method for calibrating a mass spectrometer. The computer-implemented method includes receiving, using a processor, a data set including a cathodic spacer MS data, where the cathodic spacer MS data includes at least one mass spectrum from the cathodic spacer; where the cathodic spacer has a known mass spectral profile. The computer-implemented method also includes where an analyte mixture including an ampholyte and the cathodic spacer has been subjected to an isoelectric focusing. The computer-implemented method also includes comparing, using the processor, the cathodic MS data to the known mass spectral profile to generate a set of corresponding values. The computer-implemented method also includes adjusting, using the processor, one or more mass spectrometer calibration parameters based on the set of corresponding values.

In an aspect, adjusting the one or more MS calibration parameters comprises adjusting one or more parameters for post-acquisition data recalibration.

In an aspect, the analyte mixture has been mobilized prior to introduction into a mass spectrometer.

In an aspect, the analyte mixture further includes a sample. In another aspect, the sample includes one or more biomolecules.

In an aspect, the method further includes receiving a sample MS data includes at least one mass spectrum of the sample. In an aspect, the method further includes adjusting the sample MS data based on the adjusted one or more mass spectrometer calibration parameters.

In an aspect, the cathodic spacer MS data is collected as a separate data set from the sample MS data. In an aspect, the cathodic spacer MS data and the sample MS data are collected as part of a combined data set.

In an aspect, a portion of the combined data set is used for comparing the cathodic spacer MS data with the known mass spectral profile of the cathodic spacer.

In an aspect, the cathodic spacer MS data and/or the combined data set is collected using a single mass range. In another aspect, the single mass range starts at a value above an m/z of at least one of the ampholytes.

In an aspect, the cathodic spacer MS data and/or the combined data set is collected using two or more mass ranges. In another aspect, the two or more mass ranges include a first mass range and a second mass range, wherein the second mass range starts at a value above an m/z of at least one of the ampholytes; and wherein the first mass range starts at a lower value than the value at which the second mass range starts.

In an aspect, the single mass range or at least one of the two or more mass ranges starts at about 2000 m/z units. In another aspect, at least one of the two or more mass ranges starts at about 400 m/z units. In yet another aspect, at least one of the two or more mass ranges starts at about 0 m/z units.

In an aspect, the cathodic MS data is collected using different declustering potential, collision energy, and/or time bins to sum as compared to the sample MS data.

In an aspect, the cathodic spacer is a basic amino acid. In another aspect, the amino acid is arginine, histidine, or lysine. In a further aspect, the amino acid is arginine.

In an aspect, the cathodic spacer MS data includes at least one mass spectrum from one or more multimers of arginine.

In an aspect, the cathodic spacer MS data includes m/z peaks that have an intensity greater than about 800 counts per sec (cps), alternatively greater than about 900 cps, alternatively greater than about 1000 cps, alternatively greater than about 1100 cps, or alternatively greater than about 1200 cps. In an aspect, the known mass spectral profile of the cathodic spacer includes two or more m/z peaks.

### BRIEF DESCRIPTION OF THE DRAWINGS

Aspects of the present disclosure will now be described, by way of example only, with reference to the attached Figures, wherein:

FIG. 1 is a schematic diagram of a microfluidic icIEF system with an integrated ESI sprayer.

FIG. 2 is a flow chart depicting a method for calibrating an isoelectric focusing-mass spectrometry system according to an aspect of this disclosure.

FIG. 3 is a flow chart depicting a computer-implemented method for calibrating a mass spectrometer according to an aspect of this disclosure.

FIG. 4A shows various arginine multimers according to an aspect of this disclosure. FIG. 4B shows an MS spectrum for various multimers of arginine.

FIG. 5A shows an MS data acquisition according to an aspect of this disclosure. FIG. 5B shows an MS data acquisition according to an aspect of this disclosure. FIG. 5C shows a visualization of the MS data acquired according to an aspect of this disclosure. FIG. 5D shows a visualization of the MS data acquired according to an aspect of this disclosure.

FIG. 6A is a 3-D plot of an analyte mixture including at least one ampholyte, a cathodic spacer, and a sample analyzed according to an aspect of this disclosure with about 440 m/z mass cut-off applied. FIG. 6B illustrates a 3-D plot of the analyte mixture of FIG. 6A with a 2000 m/z mass cut-off applied.

FIG. 7 is a 3-D plot of an analyte mixture including at least one ampholyte, arginine, and a sample analyzed according to an aspect of this disclosure.

### DETAILED DESCRIPTION

Unless otherwise defined, all technical and scientific terms used herein have the same meaning as commonly understood by one of ordinary skill in the art to which the methods described herein belong.

The singular form “a,” “an” and “the” include plural referents unless the context clearly dictates otherwise. These articles refer to one or to more than one (i.e., to at least one). The term “and/or” means any one or more of the items in the list joined by “and/or.” As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z.”

The term “about” as used in connection with a numerical value throughout the specification and the claims denotes an interval of accuracy, familiar and acceptable to a person skilled in the art. In general, such interval of accuracy is +/−10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The term “exemplary” means serving as a non-limiting example, instance, or illustration. As utilized herein, the terms “e.g.,” and “for example” set off lists of one or more non-limiting aspects, examples, instances, or illustrations

Isoelectric focusing (or “electrofocusing”) is a technique for separating molecules by differences in their isoelectric point (pI), i.e., the pH at which the molecules have a net zero charge. For this method, pH gradient may be formed, e.g., via use of ampholytes (amphoteric electrolytes). Anodic and/or cathodic spacers (stabilizers) can also be used—e.g., to prevent drifts of the pH gradients and/or block the ends of a fluid channel in which the electrofocusing is performed. For example, an anlyte mixture may comprise ampholyte(s) and/or spacer(s) and may be subjected to isoelectric focusing in a fluid channel by applying an electric field across the channel.

Once the electrical field is applied, a protein (or other molecule) that is in a pH region below its isoelectric point (pI) will be positively charged and so will migrate towards the cathode (i.e., the negatively charged electrode). The protein's overall net charge will decrease as it migrates through a gradient of increasing pH (due, for example, to protonation of carboxyl groups or other negatively charged functional groups) until it reaches the pH region that corresponds to its pI, at which point it has no net charge and so migration ceases. As a result, a mixture of proteins separates based on their relative content of acidic and basic residues and becomes focused into sharp stationary bands with each protein positioned at a point in the pH gradient corresponding to its pI. The technique is capable of high resolution, with proteins differing by a single charge being fractionated into separate bands. In some embodiments, isoelectric focusing may be performed in a separation channel that has been permanently or dynamically coated, e.g., with a neutral and hydrophilic polymer coating, to eliminate electroosmotic flow (EOF).

The pH gradient used for capillary isoelectric focusing techniques may be generated through the use of ampholytes, i.e., amphoteric molecules that contain both acidic and basic groups and that exist mostly as zwitterions within a certain range of pH. The electric field across a fluid channel used in isoelectric focusing may be generated by the use of electrodes and electrolyte solutions. The portion of the electrolyte solution on the anode side of the fluid channel is known as an “anolyte”. That portion of the electrolyte solution on the cathode side of the fluid channel is known as a “catholyte”. A variety of electrolytes may be used in the disclosed methods and devices including, but not limited to, phosphoric acid, sodium hydroxide, ammonium hydroxide, glutamic acid, lysine, formic acid, dimethylamine, triethylamine, acetic acid, piperidine, diethylamine, and/or any combination thereof. The electrolytes may be used at any suitable concentration, such as 0.0001%, 0.001%, 0.01%, 0.1%, 1%, 10%, 20%, 30%, 40%, 50%, 60%, 70%, 80%, 90%, etc. The concentration of the electrolytes may be at least 0.0001%, 0.001%, 0.01%, 0.1%, 1%, 10%, 20%, 30%, 40%, 50%, 60%, 70%, 80%, 90%. The concentration of the electrolytes may be at most 90%, 80%, 70%, 60%, 50%, 40%, 30%, 20%, 10%, 1%, 0.1%, 0.01%, 0.001%, 0.0001%.

Devices and methods for integration of imaged capillary isoelectric focusing with mass spectrometry (icIEF-MS) have been previously described in, for example, published PCT Patent Application Publication No. WO 2017/095813, and U.S. Patent Application Publication No. US 2017/0176386, which are hereby incorporated by reference for all purposes. These applications describe, among other things, systems for performing imaged separation in conjunction with MS analysis. Such microfluidic systems represent a significant advancement in biologics characterization.

The analysis of analytes (e.g., via icIEF-MS) can be a multistep process. In an embodiment, the process may begin with separation and focusing of analytes to their isoelectric points (pIs). The process may be followed by mobilization (accelerating separated ions towards the mass spectrometer and into an electrospray). To enable monitoring throughout certain or all of the stages of the process, ultraviolet (UV) absorbance detection or other modes of detection may be utilized. In some embodiments, the mobilization may be followed by ionization for the mass spectrometry analysis. In some embodiments, ionization may be performed using a microfluidic device that is also used for isoeclic focusing and mobilization.

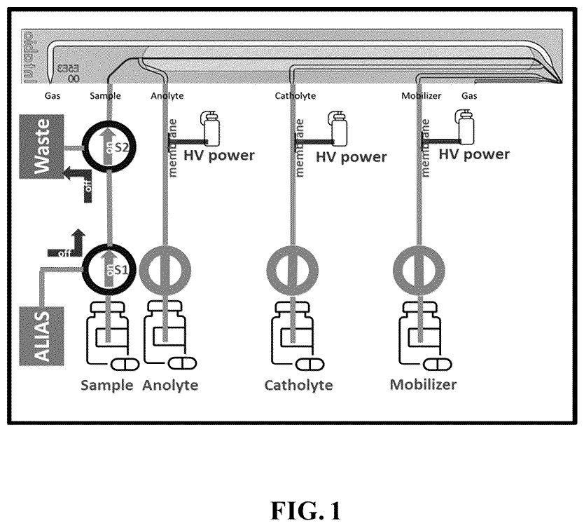

FIG. 1 is a schematic diagram of an example of an icIEF system. An analyte mixture can be introduced into a fluid channel (via an inlet labeled with the word “sample”). An electric field can be applied across the fluid channel (e.g., via anolyte and catholyte channels) to separate an analyte mixture via isoelectric focusing. The focused analyte mixture can be mobilized via introduction of a mobilizer (and, e.g., applying a field between anolyte and mobilizer channels) and further expelled into a mass spectrometer from a single orifice via electrospray ionization.

An absorbance detection (e.g., UV absorbance) may be utilized to monitor isoelectric focusing and/or mobilization (e.g., in a fluid channel). While the imaged UV trace may give pI and relative quantitation of the various proteins in the sample, there is still a need to be able to identify the protein using a mass spectrometer. This is achieved by mobilizing the proteins. During mobilization, the pH at the catholyte end starts to drop, and the protein will start to carry charges which cause it to migrate toward the mobilizer junction. Once it reaches the mobilizer junction, it will be carried by the flowing mobilizer out of the microfluidic chip and sprayed into a mass spectrometer interface.

Mass calibration of the mass spectrometric detector is necessary for sufficiently accurate data for some downstream data analysis workflows. A significant drift may be observed without periodic calibration. The onboard chromatography data system (CDS) system has limitations to use as a periodic recalibrant when the system has a secondary ion source mounted on it, like the isoelectric focusing system described. Unmounting and using the CDS is onerous and not viable during automated runs. MS calibration can performed, e.g., by adjusing one or more MS calibration parameters. In some embodiments, adjusting one or more parameters for post-acquisition data recalibration is done. Post-acquisition data recalibration may require the end user to follow up and adjust the data files after they were acquired. In some embodiments, adjusting of one or more calibration parameters for a mass spectrometer is done. For example, periodic automatic calibration runs using an isoelectric focusing system could be done.

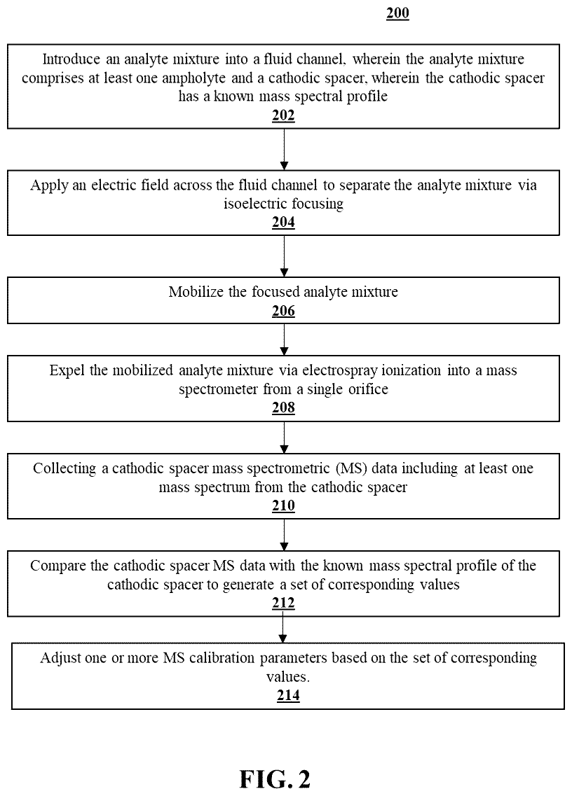

One general aspect includes a method for calibrating an isoelectric focusing-mass spectrometry using a cathodic spacer 200 and is depicted in FIG. 2. In one aspect, as part of the separation process, the method also includes introducing an analyte mixture into a fluid channel, where the analyte mixture includes at least one ampholyte and a cathodic spacer, where the cathodic spacer has a known mass spectral profile 202. The analyte mixture may also include a sample. The sample may include one or more biomolecules, for example, but not limited to, proteins, antibodies, or biologics.

The cathodic spacer may be an organic compound that will form clusters readily visible on the mass spectrometry at a relatively consistent time point within an integrated operation. The cathodic spacer may be, for example, a basic amino acid. Non-limiting examples include arginine, histidine, or lysine.

The method also includes applying an electric field across the fluid channel to separate the analyte mixture via isoelectric focusing 204. Due to the cathodic spacers' high pI the compound will elute first. The cathodic spacer also ensures that the other components of the analyte mixture (e.g., sample and/or at least one ampholyte) stay within the fluid channel.

After the components in the analyte mixture are focused, the method further includes mobilizing the focused analyte mixture 206 and expelling the mobilized analyte mixture via electrospray ionization into a mass spectrometer from a single orifice 208. In some non-limiting aspects, the single orifice is in line with the fluid channel. In another non-limiting aspect, the method includes correlating the separated analyte mixture peaks detected by imaging the fluid channel or a portion thereof with MS data for the separated analyte mixture. In this aspect, the fluid channel or a portion thereof is imaged during or after the isoelectric focusing separation and/or mobilization.

The use of the cathodic spacer as a calibrant allows a user to maintain accurate mass information and calibrate based on every injection. The method also includes collecting cathodic spacer mass spectrometric (MS) data, including at least one mass spectrum from the cathodic spacer 210, and comparing the cathodic spacer MS data with the known mass spectral profile of the cathodic spacer to generate a set of corresponding values 212.

The method also includes adjusting one or more MS calibration parameters based on the set of corresponding values 214. In a non-limiting aspect, this adjustment may include adjusting one or more calibration parameters for a mass spectrometer. In some embodiments, adjusting the one or more MS calibration parameters may include adjusting one or more parameters for post-acquisition data recalibration.

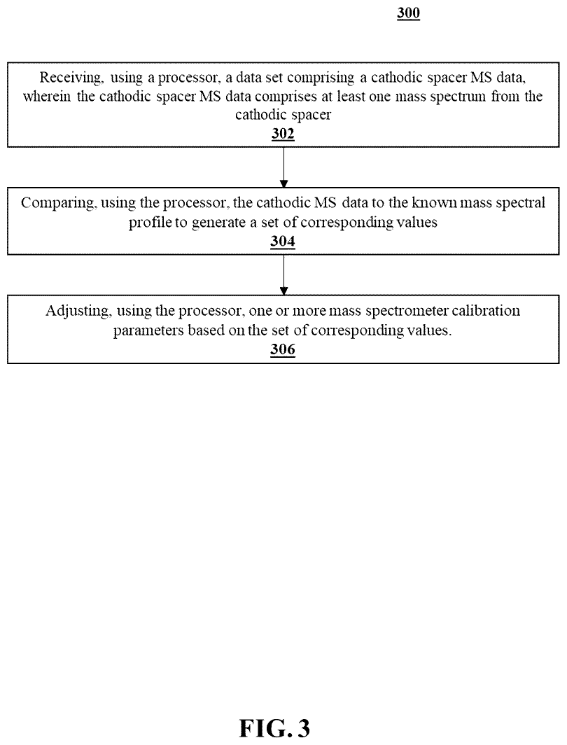

Other aspects of the disclosure include a computer-implemented method for calibrating a mass spectrometer 300, which is depicted in FIG. 3. The computer-implemented method includes receiving, using a processor, a data set including a cathodic spacer MS data, where the cathodic spacer MS data includes at least one mass spectrum from the cathodic spacer; where the cathodic spacer has a known mass spectral profile 302. In some aspects of the method, the analyte mixture, including an ampholyte and the cathodic spacer, has been subjected to an isoelectric focusing. In some aspects, the analyte mixture has also been mobilized prior to its introduction into a mass spectrometer. The computer-implemented method also includes comparing, using the processor, the cathodic MS data to the known mass spectral profile to generate a set of corresponding values 304. The method additionally includes adjusting, using the processor, one or more mass spectrometer calibration parameters based on the set of corresponding values 306.

Various MS calibration parameters can be adjusted. In some aspects, the calibration may include the creation of a calibration curve using the set of corresponding values. In some aspects, it may involve slope and/or delay time adjustment. In some aspects, post-acquisition data recalibration may be done in similar way and/or using similar parameters and techniques, but applied retroactively, on the already collected data.

In some aspects, the expected m/z of a cathodic spacer is provided, and the cathodic spacer MS data is generated and compared to the expected m/z. The MS calibration parameters were then adjusted. The known mass spectral profile of the cathodic spacer may comprise two or more m/z peaks.

An advantage of this method is that the MS calibration parameters can be adjusted in real-time (e.g., during an isoelectric focusing-mass spectrometry run) or post-acquisition. These parameters may be adjusted at various intervals. For example, the one or more MS calibration parameters at least every other introduction, alternatively at least every introduction, or alternatively at least every 30 minutes, alternatively at least every hour.

In one aspect, the cathodic spacer forms multimers when subjected to electrospray ionization (ESI). In this aspect, the cathodic spacer MS data comprises at least one mass spectrum from one or more multimers of the cathodic spacer. In a non-limiting example, the cathodic spacer MS data comprises at least one mass spectrum from one or more multimers of arginine. The multimers (or clusters) are various charge states of the cathodic spacer. Table 1 is an exemplary list of various multimers of arginine (along with monomers), wherein arginine has a molecular weight (MW) of appx. 174.112 and hydrogen has a MW of appx. 1.00783.

TABLE 1

| M + H | MW | M + 2H | MW |
|---|---|---|---|
| [1Arg + 1H]⁺ | 175.1195 | [1Arg + 2H]⁺⁺ | 88.06366 |
| [2Arg + 1H]⁺ | 349.2312 | [2Arg + 2H]⁺⁺ | 175.1195 |
| [3Arg + 1H]⁺ | 523.3429 | [3Arg + 2H]⁺⁺ | 262.1753 |
| [4Arg + 1H]⁺ | 697.4545 | [4Arg + 2H]⁺⁺ | 349.2312 |
| [5Arg + 1H]⁺ | 877.3705 | [5Arg + 2H]⁺⁺ | 436.287 |
| [6Arg + 1H]⁺ | 1045.678 | [6Arg + 2H]⁺⁺ | 523.3429 |
| [7Arg + 1H]⁺ | 1219.79 | [7Arg + 2H]⁺⁺ | 610.3987 |
| [8Arg + 1H]⁺ | 1393.901 | [8Arg + 2H]⁺⁺ | 697.4545 |
| [9Arg + 1H]⁺ | 1568.013 | [9Arg + 2H]⁺⁺ | 784.5104 |
| [10Arg + 1H]⁺ | 1742.125 | [10Arg + 2H]⁺⁺ | 871.5662 |
| [11Arg + 1H]⁺ | 1916.236 | [11Arg + 2H]⁺⁺ | 958.622 |
| [12Arg + 1H]⁺ | 2090.348 | [12Arg + 2H]⁺⁺ | 1045.678 |
| [13Arg + 1H]⁺ | 2264.46 | [13Arg + 2H]⁺⁺ | 1132.734 |
| [14Arg + 1H]⁺ | 2438.571 | [14Arg + 2H]⁺⁺ | 1219.79 |
| [15Arg + 1H]⁺ | 2612.683 | [15Arg + 2H]⁺⁺ | 1306.845 |
| [16Arg + H]⁺ | 2786.795 | [16Arg + 2H]⁺⁺ | 1393.901 |
| [17Arg + 1H]⁺ | 2960.906 | [17Arg + 2H]⁺⁺ | 1480.957 |
| [18Arg + 1H]⁺ | 3135.018 | [18Arg + 2H]⁺⁺ | 1568.013 |
| [19Arg + 1H]⁺ | 3309.13 | [19Arg + 2H]⁺⁺ | 1655.069 |
| [20Arg + 1H]⁺ | 3483.241 | [20Arg + 2H]⁺⁺ | 1742.125 |
| [21Arg + 1H]⁺ | 3657.353 | [21Arg + 2H]⁺⁺ | 1829.18 |
| [22Arg + 1H]⁺ | 3831.465 | [22Arg + 2H]⁺⁺ | 1916.236 |
| [23Arg + 1H]⁺ | 4005.576 | [23Arg + 2H]⁺⁺ | 2003.292 |
| [24Arg + 1H]⁺ | 4179.688 | [24Arg + 2H]⁺⁺ | 2090.348 |
| [25Arg + 1H]⁺ | 4353.8 | [25Arg + 2H]⁺⁺ | 2177.404 |
| [26Arg + IH]⁺ | 4527.911 | [26Arg + 2H]⁺⁺ | 2264.46 |
| [27Arg + 1H]⁺ | 4702.023 | [27Arg + 2H]⁺⁺ | 2351.515 |
| [28Arg + 1H]⁺ | 4876.135 | [28Arg + 2H]⁺⁺ | 2438.571 |
| | | [29Arg + 2H]⁺⁺ | 2525.627 |
| | | [30Arg + 2H]⁺⁺ | 2612.683 |

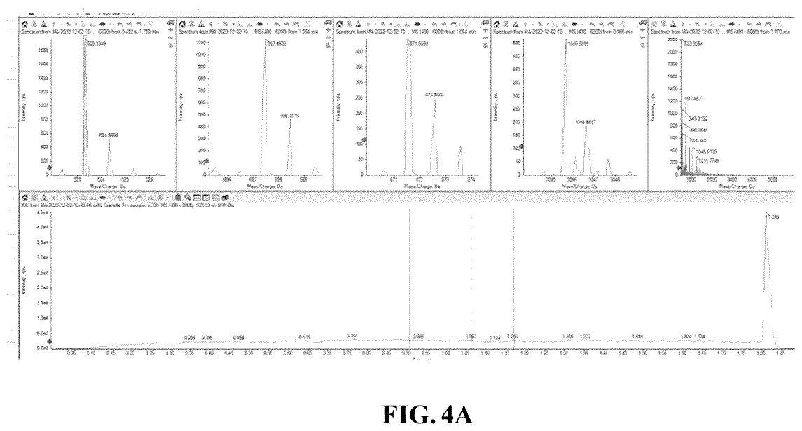

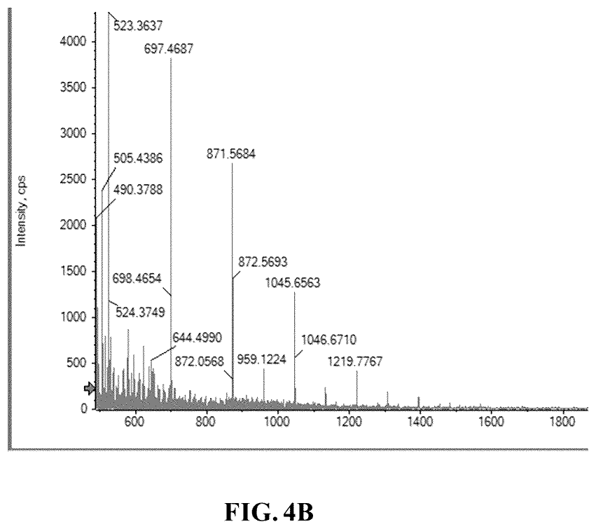

The multimers used for calibration can be selected based on several factors, including identifying multimers with the least redundancy (i.e., a single and singly charged would have the same monoisotopic m/z as a double doubly charged, so it would be ideal to avoid) or identifying multimers with the highest baseline intensity. As shown in FIGS. 4A and 4B, multimers of arginine with the highest baseline intensity were selected for calibration.

The cathodic spacer MS data may include m/z peaks with an intensity greater than about 800 counts per sec (cps), alternatively greater than about 900 cps, alternatively greater than about 1000 cps, alternatively greater than about 1100 cps, or alternatively greater than about 1200 cps. If the cathodic spacer MS data includes multimers, m/z peaks of varying intensities may be present.

The method also includes collecting a sample MS data comprising at least one mass spectrum of the sample. The sample MS data can be adjusted based on the one or more MS calibration parameters. While the cathodic spacer MS data and the sample MS data may be collected as part of a combined data set, the method also allows for two runs where the cathodic spacer MS data is collected as a separate data set from the sample MS data.

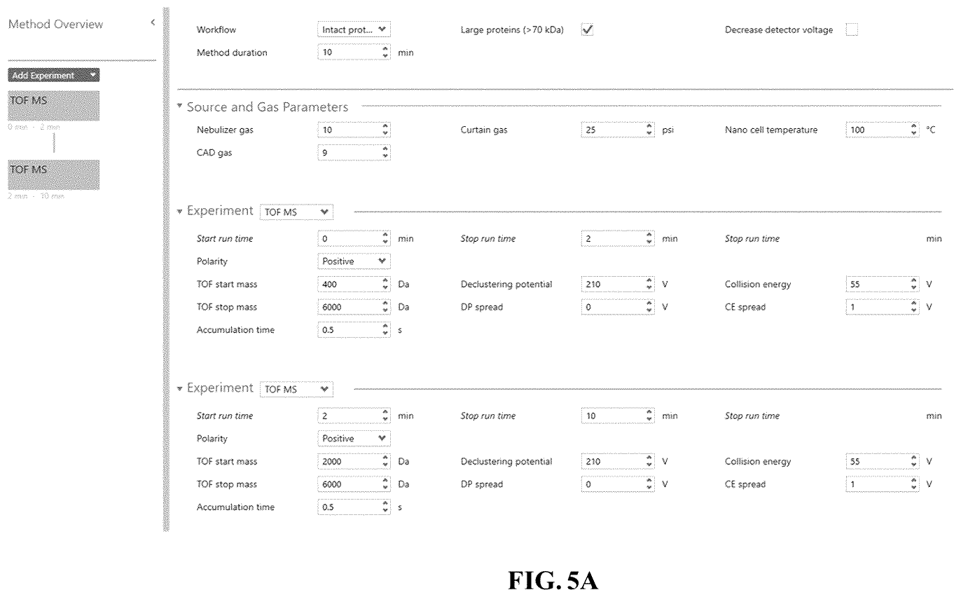

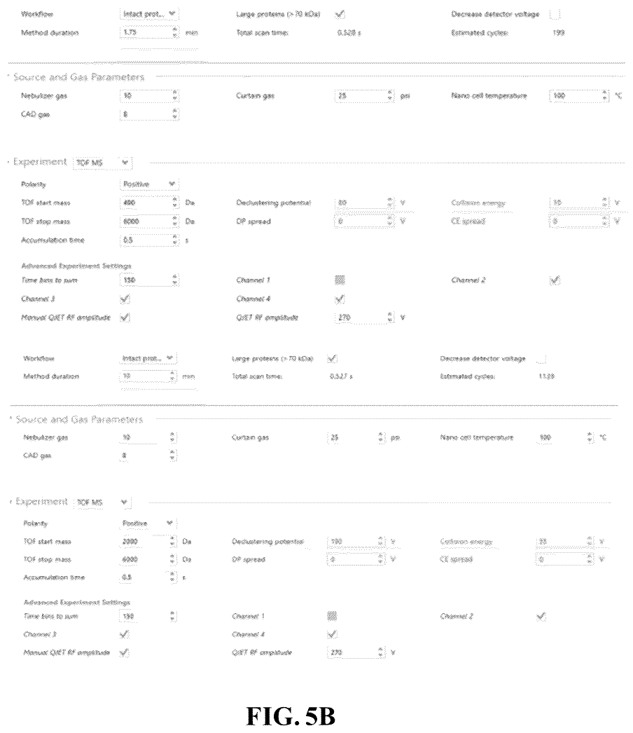

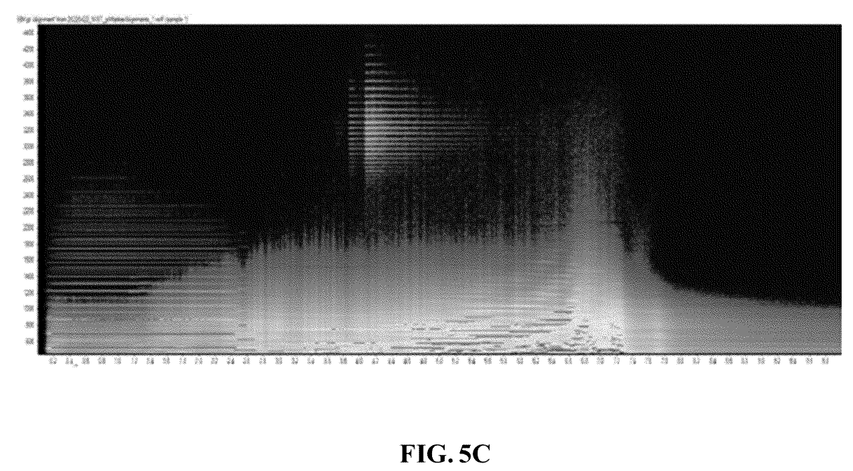

As shown in FIG. 5A, the cathodic MS data may be collected using the same declustering potential, collision energy, and/or time bins to sum as compared to the sample MS data. As shown in FIG. 5B, the cathodic MS data may be collected using different declustering potential, collision energy, and/or time bins to sum as compared to the sample MS data. In another example, time bins to sum coud be 10 for the cathodic MS data (e.g., for arginine) and/or 150 for the sample MS data (e.g., for mAb). In an aspect, using a higher declustering potential or collision energy for the larger sample molecules ensures there are no extra waters clustered on the biomolecule. However, the multimers formed will be destroyed if the same declustering potential or collision energy is used for the smaller cathodic spacer.

In another example, using a declustering potential, collision energy, and/or time bins to sum that is lower than the sample MS data acquisition for the acquisition of the cathodic spacer MS data may result in better resolved cathodic spacer m/z peaks. This may allow for improved scanning of the m/z peaks and increased accuracy when selecting the apex value.

In an aspect, the cathodic spacer MS data and the sample MS data maybe be collected as part of a combined data set. An example of a method wherein a cathodic spacer MS data and a sample MS data are collected as part of a combined data set is shown in FIG. 5A. An example of a combined data set is shown in FIG. 5C. In an aspect, a portion of the combined data set is used for comparing the cathodic spacer MS data with the known mass spectral profile of the cathodic spacer. In another aspect, the cathodic spacer MS data may be collected as a separate data set from the sample MS data. An example of methods wherein a cathodic spacer MS data and a sample MS data are collected as part of a separate data sets is shown in FIG. 5B. An example of a vizualization of separate data sets for a cathodic spacer MS data (labeled as “2 MIN CAL”) and a sample MS data (labled as “PROPER RUN”) in FIG. 5D.

In one aspect, the cathodic spacer MS data and/or the combined data set is collected using a single mass range or two or more mass ranges. Non-limiting examples of the cathodic spacer MS data and/or the combined data set collected using two or more mass ranges is showin in FIGS. 5A and 5B. In an example, the mass range may start at a value above an m/z of at least one of the ampholytes. When the data is collected using two or more mass ranges, a second mass range starts at a value above an m/z of at least one of the ampholytes, and a first mass range starts at a lower value than the value at which the second mass range starts.

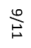

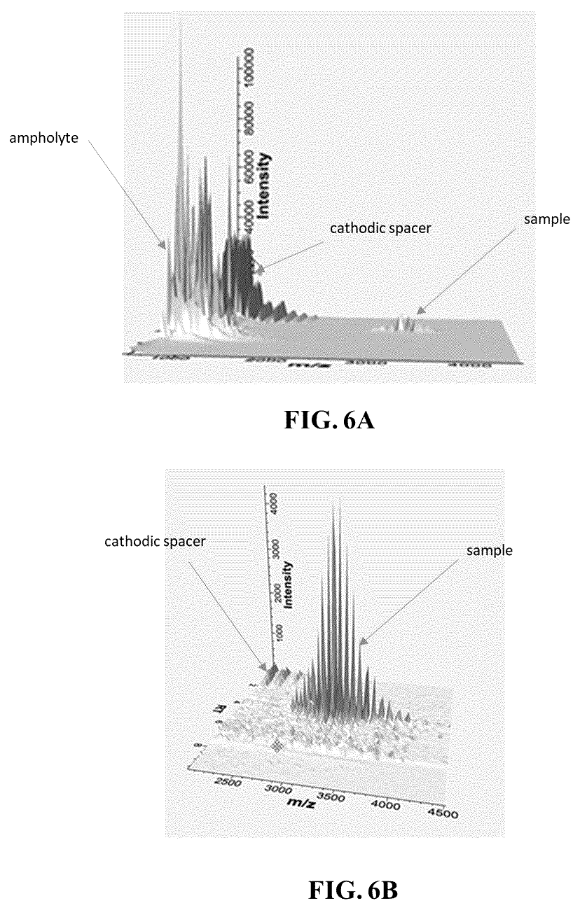

In another non-limiting example, the single mass range or at least one of the two or more mass ranges starts at about 2000 m/z units, alternatively about 400 m/z units, alternatively about 0 m/z units. As shown in FIGS. 6A and 6B, by collecting data using a mass range cut off, some of the noise (e.g., the ampholyte(s)) can be eliminated, which proves beneficial when analyzing samples with low concentrations.

### Example: Exemplary Isoelectric Focusing-Mass Spectrometry Method

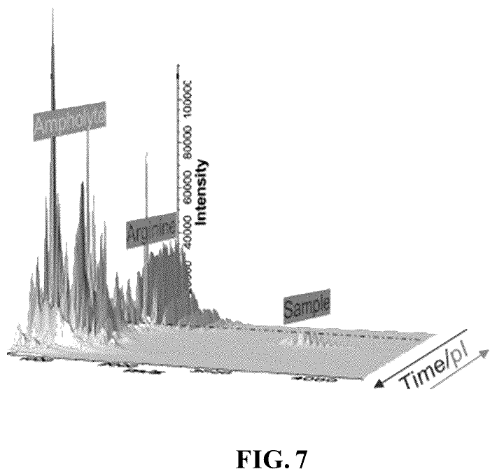

The device depicted in FIG. 1 may be used for the method described in this example. A prime script may be initiated to prime channels with the corresponding solutions (e.g., an analyte mixture, anolyte, catholyte, and/or mobilizer solutions). An analyte mixture, including an ampholyte, arginine, and a sample, is introduced into a fluid channel. An electric field is applied across the first fluid channel to separate the analyte mixture via isoelectric focusing. A mobilization script is initiated and the MS acquisition is triggered. During the mobilization script, a first MS acquisition is performed corresponding to the length of the arginine peak. After that time interval (e.g., appx. 2 min), a second MS acquisition is performed. After the mobilization script is complete, the wash script is initiated. The script sequence can be iterated as many times as chosen injections. FIG. 7 depicts MS data obtained using this method.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

It will be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for calibrating an isoelectric focusing-mass spectrometry system comprising:
introducing an analyte mixture into a fluid channel, wherein the analyte mixture comprises at least one ampholyte and a cathodic spacer, wherein the cathodic spacer has a known mass spectral profile;
applying an electric field across the fluid channel to separate the analyte mixture via isoelectric focusing;
mobilizing the focused analyte mixture;
expelling the mobilized analyte mixture via electrospray ionization into a mass spectrometer from a single orifice;
collecting a cathodic spacer mass spectrometric (MS) data comprising at least one mass spectrum from the cathodic spacer;
comparing the cathodic spacer MS data with the known mass spectral profile of the cathodic spacer to generate a set of corresponding values; and
adjusting one or more MS calibration parameters based on the set of corresponding values.

2. The method of claim 1, wherein adjusting the one or more MS calibration parameters comprises adjusting one or more calibration parameters for a mass spectrometer and/or for post-acquisition data recalibration.

3. (canceled)

4. The method of claim 1, wherein the analyte mixture further comprises a sample comprising one or more biomolecules.

5. (canceled)

6. The method of claim 4, further comprising:
collecting a sample MS data comprising at least one mass spectrum of the sample, wherein the cathodic spacer MS data is collected using different declustering potential, collision energy, and/or time bins to sum as compared to the sample MS data; and
adjusting the sample MS data based on the adjusted one or more MS calibration parameters.

7. (canceled)

8. The method of claim 6, wherein cathodic spacer MS data is collected as a separate data set from the sample MS data or as part of a combined data set.

9. (canceled)

10. The method of claim 8, wherein a portion of the combined data set is used for comparing the cathodic spacer MS data with the known mass spectral profile of the cathodic spacer.

11. The method of claim 8, wherein the cathodic spacer MS data and/or the combined data set is collected using a single mass range or using two or more mass ranges, or wherein the single mass range starts at a value above an m/z of at least one of the ampholytes and/or wherein the single mass range or at least one of the two or more mass ranges starts at about 2000, about 400, or about 0 m/z units.

12. (canceled)

13. (canceled)

14. The method of claim 11, wherein the two or more mass ranges comprise a first mass range and a second mass range,
and
wherein the first mass range starts at a lower value than the value at which the second mass range starts.

15. (canceled)

16. (canceled)

17. (canceled)

18. (canceled)

19. The method of claim 1, wherein the single orifice is in line with the fluid channel.

20. The method of claim 1, further comprising correlating the separated analyte mixture peaks detected by imaging of the fluid channel or a portion thereof with an MS data for the separated analyte mixture.

21. The method of claim 1, wherein the fluid channel or a portion thereof is imaged during or after the isoelectric focusing separation and/or mobilization.

22. The method of claim 1, wherein the cathodic spacer forms multimers when subjected to electrospray ionization (ESI).

23. The method of claim 1, wherein the cathodic spacer is a basic amino acid that is arginine, histidine, or lysine.

24. (canceled)

25. (canceled)

26. The method of claim 23, wherein the cathodic spacer MS data comprises at least one mass spectrum from one or more multimers of arginine or m/z peaks that have an intensity greater than about 800 counts per sec (cps), alternatively greater than about 900 cps, alternatively greater than about 1000 cps, alternatively greater than about 1100 cps, or alternatively greater than about 1200 cps.

27. (canceled)

28. The method of claim 1, wherein the known mass spectral profile of the cathodic spacer comprises two or more m/z peaks.

29. The method of claim 1, wherein the method further comprises:
excluding a direct injection of a calibrant into a mass spectrometer; and/or
adjusting the one or more MS calibration parameters at least every other introduction.

30. (canceled)

31. A computer-implemented method for calibrating a mass spectrometer comprising:
receiving, using a processor, a data set comprising a cathodic spacer MS data,
wherein the cathodic spacer MS data comprises at least one mass spectrum from the cathodic spacer; wherein the cathodic spacer has a known mass spectral profile,
and wherein an analyte mixture comprising an ampholyte and the cathodic spacer has been subjected to an isoelectric focusing;
comparing, using the processor, the cathodic MS data to the known mass spectral profile to generate a set of corresponding values; and
adjusting, using the processor, one or more MS calibration parameters based on the set of corresponding values.

32. The method of claim 31, wherein adjusting the one or more MS calibration parameters comprises adjusting one or more parameters for post-acquisition data recalibration.

33. The method of claim 31, wherein the analyte mixture has been mobilized prior to introduction into a mass spectrometer and/or wherein the analyte mixture further comprises a sample that comprises one or more biomolecules.

34. (canceled)

35. (canceled)

36. The method of claim 34, further comprising:
receiving a sample MS data comprising at least one mass spectrum of the sample; and/or
adjusting the sample MS data based on the adjusted one or more MS calibration parameters.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)
